(19) United States
(12) Patent Application Publication
(10) Pub. No.: US 2021/0128676 A1
(43) Pub. Date: May 6, 2021

(54) METHOD OF TREATING PATIENTS WITH HEPATORENAL SYNDROME TYPE 1 AND LOW MEAN ARTERIAL PRESSURE

(71) Applicant: Mallinckrodt Hospital Products IP Unlimited Company, Dublin 15 (IE)

(72) Inventors: Khurram Jamil, Yardley, PA (US); Stephen Chris Pappas, Lebanon, NJ (US); Peter Teuber, Lebanon, NJ (US)

(21) Appl. No.: 17/083,409

(22) Filed: Oct. 29, 2020

Related U.S. Application Data
(60) Provisional application No. 62/928,152, filed on Oct. 30, 2019.

(51) Int. Cl. A61K 38/095; A61K 38/38

(52) U.S. Cl. CPC A61K 38/095; A61K 9/0019; A61K 38/38

(57) ABSTRACT

The principles and embodiments of the present disclosure relate to methods of increasing survival of a patient having type 1 hepatorenal syndrome (HRS-1) and low mean arterial pressure (MAP). The methods may include identifying a patient having HRS-1 that has a baseline MAP of less than 65 mmHg, and administering, to the patient, an amount of terlipressin effective to treat the HRS-1 in the patient. In other aspects, the method may include administering an effective dose of terlipressin to a patient in need thereof every 6 hours by intravenous (IV) bolus injection over 2 minutes, where the dose is sufficient to yield an increase in MAP and decrease in heart rate in the patient. The patient may not have overt sepsis, septic shock, or uncontrolled infection.

### CLAIM OF PRIORITY

This application claims priority under 35 USC § 119(e) to U.S. Patent Application Ser. No. 62/928,152, filed on Oct. 30, 2019, the entire contents of which are hereby incorporated by reference.

### INCORPORATION OF SEQUENCE LISTING

A computer readable text file, entitled “664953_SequenceListing_ST25.txt” created on or about 28 Oct. 2020, with a file size of about 1 kilobyte contains the sequence listing for this application and is hereby incorporated by reference in its entirety.

### TECHNICAL FIELD

Principles and embodiments of the present disclosure relate generally to methods of treating patients with type-1 hepatorenal syndrome having a low mean arterial pressure.

### BACKGROUND

Hepatorenal Syndrome Type-1 (HRS Type 1 or HRS-1) is the development of acute kidney failure in patients with late-stage liver cirrhosis in the absence of any other cause. It is characterized by rapid onset of renal failure with a high mortality rate that exceeds 80% with within three months. Renal failure is an identified complication of cirrhosis of the liver; and, acute renal failure is known to have poor prognosis for patients with cirrhosis of the liver. In various instances, the renal failure may be caused by hypovolemia, hepatorenal syndrome without ongoing infection, or hepatorenal syndrome with an ongoing infection. Unfortunately, patients with HRS Type-1 may die from renal failure while waiting for a liver transplant. Currently, there is no way of determining which patients could maximally benefit from terlipressin treatment to reverse HRS Type-1.

Hepatorenal Syndrome (HRS) is indicated by low glomerular filtration rate due to renal vasoconstriction, splanchnic and peripheral arterial vasodilatation producing decreased vascular resistance, and portal hypertension. HRS is indicated by cirrhosis with ascites, serum levels of creatinine>133 μmol/l (1.5 mg/dL), no improvement of serum levels of creatinine (decrease to a level of ≤133 μmol/l) after at least 2 days of diuretic withdrawal and volume expansion with albumin, and the absence of shock and parenchymal kidney disease. HRS Type 1 is indicated by doubling of the initial serum levels of creatinine to >226 μmol/l (2.56 mg/dL) in <2 weeks.

Normal creatinine levels range from 0.7 to 1.3 mg/dL in men and 0.6 to 1.1 mg/dL in women. One mg/dl of creatinine equals 88.4 μmol/l.

Certain mechanisms that work to maintain effective arterial blood volume and relatively normal arterial pressure in patients with cirrhosis, however, affect kidney function, such as sodium and solute-free water retention, which can lead to ascites and edema, and to renal failure by causing intrarenal vasoconstriction and hypoperfusion. Ascites can result from the combination of portal hypertension and splanchnic arterial vasodilation that alters intestinal capillary pressure and permeability, which facilitates the accumulation of the retained fluid in the abdominal cavity.

A factor contributing to ascites formation is a splanchnic vasodilation that results in a decreased effective arterial blood volume. Portal hypertension also results from increased hepatic resistance to portal blood flow in cirrhotic livers, and may induce splanchnic vasodilation. There may be a marked impairment in solute-free renal water excretion and renal vasoconstriction, which leads to HRS.

In various instances, there may be signs of hepatic decompensation including INR>1.5, ascites, and encephalopathy. Hyponatremia is also a frequent complication of patients with cirrhosis and ascites that is associated with increased morbidity.

Sepsis has been defined as a systemic inflammatory response to infection, and septic shock is sepsis complicated by either hypotension that is refractory to fluid resuscitation or by hyperlactatemia.

Low mean arterial pressure (MAP)<65 mmHg, hypotension, is common in patients with decompensated cirrhosis. Hypotension often occurs with the absence of overt shock as evidenced by hypoperfusion abnormalities (e.g. peripheral cyanosis, hypothermia, marked asthenia, pallor, obtundation not attributable to hepatic encephalopathy). This finding results from hemodynamic changes characterized by splanchnic and peripheral vasodilation related to portal hypertension and circulating factors leading to peripheral vasodilation.

Low MAP <65 mmHg has typically been associated with a poorer prognosis and a propensity to develop further complications in this group of patients. These patients are usually not treated with vasopressors for this asymptomatic hypotension alone

Terlipressin is a synthetic analogue of vasopressin having a prolonged effect, which acts as a peptidic vasopressin Vla receptor agonist. Terlipressin is a derivative of vasotocin prepared by extending the N-terminal by three amino acid residues, and used as a vasoactive drug in the management of hypotension. Terlipressin may be synthesized by coupling amino acids stepwise to one another in a liquid or solid phase with a peptide synthesizer. Terlipressin is a prodrug that slowly metabolizes to lysine-vasopressin and in this way provides prolonged biological effect. The half-life of terlipressin is 6 hours (the duration of action is 2-10 hr), as opposed to the short half-life of vasopressin, which is only 6 minutes (the duration of action is 30-60 min).

The chemical structure for terlipressin (Gly-Lys-Pro-Cys-Asn-Gln-Phe-Tyr-Cys-Gly-Gly-Gly; SEQ ID NO: 1) in an injectable formulation is show below.

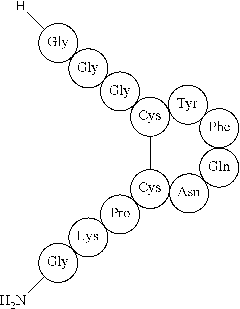

Molecular Formula: C₅₂ H₇₄ N₁₆ O₁₅ S₂

Molecular Weight: 1227.4 daltons

Appearance: Homogenous lyophilized white to off-white solid

Solubility: Clear, colorless solution in saline

Vials: Colorless glass vials containing 11 mg of a white to off-white solid, 1 mg active ingredient and 10 mg mannitol.

The active ingredient, N—[N—(N-glycylglycyl)glycyl]-8-L-lysinevasopressin, is a synthetically manufactured hormonogen of 8-lysine vasopressin, composed of 12 amino acids and having the characteristic ring structure of a cyclic nonapeptide with a disulfide bridge between the fourth and the ninth amino acid. Three glycyl-amino acids are substituted at position 1 (cysteine) of 8-lysine-vasopressin. By this N-terminal extension of 8-lysine-vasopressin the metabolic degradation rate of the active ingredient is significantly reduced, because the glycyl molecules inhibit rapid N-terminal enzymatic degradation. Terlipressin may be present in pharmaceutical compositions as a salt, diacetate salt, hydrate, and/or free base, such as terlipressin acetate or terlipressin diacetate pentahydrate.

### SUMMARY

Principles and embodiments of the present disclosure relate generally to methods of treating patients having HRS-1 by administering terlipressin to the patients to obtain reversal of the HRS-1, improved overall survival, and/or improved transplant-free survival. In one or more embodiments, a low baseline mean arterial pressure (MAP) of less than 65 mmHg provides a new and useful function of indicating a likelihood of improved response by a patient to the administration of terlipressin.

Some aspects of the disclosure relate to a method of treating HRS-1, where the method includes identifying a patient as having HRS-1; determining that the patient exhibits a mean arterial pressure of less than 65 mmHg; determining that the patient does not have uncontrolled infection, sepsis, or septic shock; determining that, because the patient exhibits a mean arterial pressure of less than 65 mmHg, the HRS-1 of the patient is likely to respond to treatment with terlipressin; and administering to the patient an amount of terlipressin effective to treat HRS-1 in the patient. The patient treated with terlipressin may experience an increased overall survival and/or transplant-free survival compared to placebo. In some examples, the terlipressin administered may be terlipressin acetate.

In additional aspects of the disclosure, a method of increasing survival of a patient having HRS-1 and low MAP includes administering an effective dose of terlipressin to a patient in need thereof about every 6 hours by intravenous (IV) bolus injection over about 2 minutes, where the dose is sufficient to yield an increase in MAP and decrease in heart rate in the patient.

In other aspects of the disclosure, a method of increasing survival of a patient having HRS-1 and low MAP includes administering an effective dose of terlipressin to a patient in need thereof about every 6 hours by intravenous (IV) bolus injection over about 2 minutes, where the dose is sufficient to yield an increase in the diastolic, systolic and MAP, and decrease in heart rate in the patient.

In further aspects of the disclosure, a method of increasing survival of a patient having HRS-1 and low MAP includes administering an effective dose of about 0.5 mg to about 2 mg terlipressin acetate to a patient in need thereof about every 4 to 10 hours by intravenous (IV) bolus injection over about 1 to 5 minutes, where the dose is sufficient to yield an increase in MAP and decrease in heart rate in the patient.

Additional aspects and features are set forth in part in the description that follows, and will become apparent to those skilled in the art upon examination of the specification or may be learned by the practice of the disclosed subject matter.

### BRIEF DESCRIPTION OF THE DRAWINGS

Further features of embodiment of the present disclosure, their nature and various advantages will become more apparent upon consideration of the following detailed description, taken in conjunction with the accompanying drawings, which are also illustrative of the best mode contemplated by the applicants, and in which like reference characters refer to like parts throughout, where:

FIG. 1 illustrates an exemplary embodiment of a terlipressin treatment protocol;

FIG. 2 is a graph of the overall survival of the two patient groups, comparing the treatment groups and placebo;

FIG. 3 is a graph of the transplant-free survival of the two patient groups, comparing the treatment groups and placebo; and

FIG. 4 illustrates an exemplary embodiment of a terlipressin treatment protocol.

### DETAILED DESCRIPTION

As used herein, the term “asymptomatic hypotension” is defined as a MAP <65 mmHg in the absence of shock.

As used herein, the term “low MAP” is defined as a MAP <65 mmHg.

As used herein, use of “terlipressin” may refer to terlipressin or salts, diacetate salts, hydrates, and/or free bases thereof. For example, use of terlipressin may include terlipressin acetate or terlipressin diacetate pentahydrate. In additional examples, terlipressin may refer to any other suitable salts or hydrates thereof or any other biologically acceptable salts or hydrates thereof.

The principles and embodiments of the present disclosure relate to methods of improving a patient's renal condition involving a treatment protocol comprising terlipressin. Accordingly, various embodiments of the present disclosure provide methods of treating a patient with terlipressin or terlipressin and albumin.

In embodiments of the present disclosure, the patient is evaluated to determine the particular disease and/or syndrome he or she may be suffering from, and beginning a treatment regimen for patients that will benefit from the administration of terlipressin.

In various embodiments, the patient has end stage liver disease complicated with acute kidney failure, such as HRS, and is treated with terlipressin.

In various embodiments, end-stage liver disease may be cirrhosis of the liver or fulminant liver failure. In various embodiments, the end-stage liver disease is complicated by impaired renal function.

An aspect of the present disclosure relates to a method of diagnosis of patients that show improved response to terlipressin treatment, as indicated by an increased probability of HRS reversal.

In one or more embodiments, the method of identifying an HRS-1 patient with an increased likelihood of responding to terlipressin treatment regimen comprises identifying a patient as having end stage live disease and impaired renal function, determining that a patient of the plurality exhibits a mean arterial pressure of less than 65 mmHg; determining that, because the patient exhibits a mean arterial pressure of less than 65 mmHg, the HRS-1 of the patient is likely to respond to treatment with terlipressin; administering to the patient an amount of terlipressin effective to treat HRS-1 in the patient.

In various embodiments, the terlipressin dosage is administered to the patient in the range of about 0.5 mg to about 2.0 mg about every 4 to 6 hours, as a series of single doses, so that the patient receives a single dose in the range of about 0.5 mg to about 2.0 mg of terlipressin followed by another single dose about 4 to 6 hours later. In various embodiments, a patient may receive about 4 to 6 doses over about a 24 hour period, where each dose is in the range of about 0.5 mg to about 2.0 mg. In various embodiments, the total dosage does not exceed about 12.0 mg over a 24 hour period.

In various embodiments, a patient, who is initially identified as having end stage liver disease, for which treatment with a vasodilator may provide an improvement in renal function, is tested to determine the extent of the patient's cirrhosis and renal failure.

The administration of terlipressin to patients with decompensated cirrhosis who are critically ill but not usually treated for asymptomatic hypotension was surprisingly effective in this group of patients. For example, lower baseline MAP unexpectedly predicted improved overall survival (OS) and transplant-free survival (TFS) in cirrhotic patients with HRS-1 treated with terlipressin.

OS and TFS may be significantly higher in patients with baseline MAP <65 mmHg treated with terlipressin compared to placebo. In comparison, patients with baseline MAP 65 mmHg may have no difference in OS or TFS between patients treated with terlipressin compared to placebo. In some examples, this effect may be independent of response to treatment with terlipressin as defined by achieving HRS reversal (serum creatinine (SCr) decrease to 1.5 mg/dL). Without being limited to any one theory, this effect may be related to the significantly increased MAP in subjects with MAP <65 mmHg treated with terlipressin.

In one or more embodiments, the terlipressin treatment protocol comprises identifying a patient having end-stage liver disease and impaired renal function, where the identified patient may benefit from a treatment comprising administration of terlipressin, determining that the patient exhibits a mean arterial pressure of less than 65 mmHg; administering to the patient a dosage of terlipressin in an amount effective to produce an improvement in renal function. An improvement in renal function is indicated by a reduction in SCr of at least 25% from baseline, reversal of HRS (defined as a decrease in SCr level to ≤1.5 mg/dl), and/or confirmed HRS reversal (defined as two serum creatinine values of ≤1.5 mg/dL at least 48 hours apart)).

In one or more embodiments, the terlipressin dosage may be in the range of about 0.5 mg to about 10 mg, or about 0.5 mg to about 5.0 mg, or about 0.5 mg to about 2.0 mg, or about 0.5 mg to about 1 mg, or about 1.0 mg to about 2.0 mg per single administration. In various embodiments, the injections may be administered intravenously as slow bolus injections over about 2 minutes, where the dose may be repeated about every four to six hours. If on day 4 of therapy (after a minimum of 10 doses), SCr had decreased, but by less than 30% from the baseline value, the dose may be increased to 2 mg every 6 hours (±30 min) (8 mg/day). The dose may not be increased if the subject had coronary artery disease; or in the clinical setting of circulatory overload, pulmonary edema, or treatment-refractory bronchospasm. In various embodiments, if dosing was interrupted due to a non-ischemic adverse event, terlipressin may be restarted at the same or lower dose (i.e., 0.5 to 1 mg q6h).

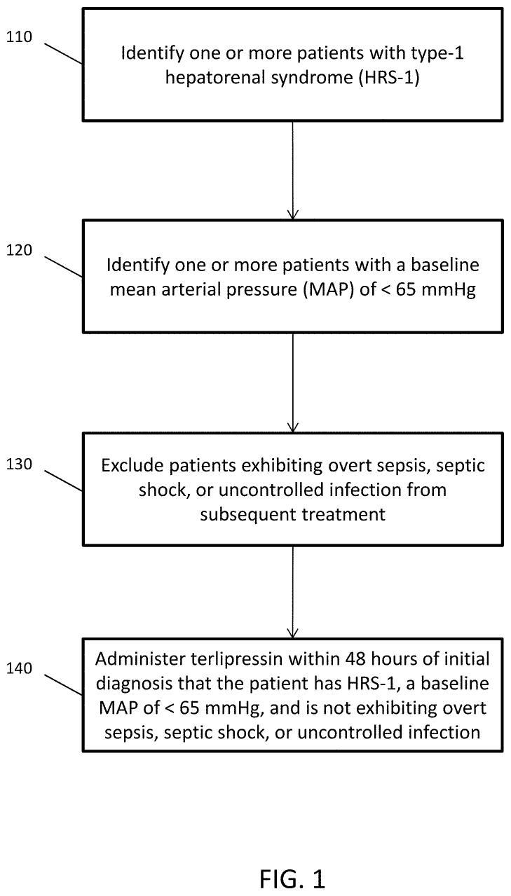

FIG. 1 illustrates an exemplary embodiment of a terlipressin treatment protocol.

Principles and embodiments of the present disclosure also relate to providing terlipressin as an IV every four to six hours to patients that have been identified with HRS-1 and low MAP.

In one or more embodiments, a patient is tested for mean arterial pressure prior to treatment.

In one or more embodiments of the disclosure, terlipressin is administered to patients presenting with a particular set of symptoms to mitigate the vasoconstriction in the kidneys, and improve renal function as indicated by a reduction in serum creatinine levels of about 1.7 mg/dL from initial baseline.

At 110, one or more patients with HRS-1 are identified. In some embodiments, one or more patients that may be presenting with end-stage liver disease are tested to determine whether they are suffering from cirrhosis with ascites, and have serum levels of creatinine>133 μmol/l. A patient identified as having HRS is further tested and/or their medical history checked to determine if the initial serum levels of creatinine have doubled to greater than 226 μmol/l in less than 2 weeks indicating type 1 HRS.

At 120, once a patient has been identified as suffering from HRS-1, the patient is tested to determine is the same patient has a baseline MAP of less than 65 mmHg.

In various embodiments, patients not identified as exhibiting a baseline MAP of less than 65 mmHg are excluded from the terlipressin treatment protocol. Patients having HRS-1 and a baseline MAP of less than 65 mmHg have surprisingly shown improved response to terlipressin treatment compared to HRS-1 patients with a baseline MAP greater than or equal to 65 mmHg, as indicated by increased overall survival and transplant-free survival.

At 130, patients that have been identified as having HRS-1 and a baseline MAP of less than 65 mmHg are tested to determine if they may also have an uncontrolled infection, sepsis, or septic shock. Patients identified as exhibiting an uncontrolled infection, sepsis, or septic shock are excluded from the terlipressin treatment protocol.

At 140, patients that have HRS-1, have a baseline MAP of less than 65 mmHg, and do not have an uncontrolled infection, sepsis, or septic shock are started on the terlipressin treatment. In one or more embodiments, the terlipressin treatment is started within 48 hours of the initial diagnosis that the patient has both HRS-1 and a baseline MAP of less than 65 mmHg. In various embodiments, in which the determination that the patient does or does not also have an uncontrolled infection, sepsis, or septic shock occurs after 48 hours of the initial diagnosis of both HRS-1 and the low MAP, the treatment protocol is started within 48 hours of the initial diagnosis, and treatment may be terminated once an uncontrolled infection, sepsis, or septic shock manifests or is determined.

In various embodiments, a baseline serum creatinine level may be determined for the patient prior to starting the administration of terlipressin to the patient; and the administration of terlipressin started within 2 days or within 3 days, or within 4 days of determining the baseline serum creatinine level. In various embodiments, the patient may be tested at least once daily within four days after starting the administration of terlipressin to determine if the patient exhibits a decrease in the serum creatinine level compared to the previously determined baseline serum creatinine level.

In one or more embodiments, the terlipressin may be administered to a patient as a slow infusion over 24 hours, wherein the dosage over the 24 hour period may be in the range of about 2.0 mg to about 12 mg. In various embodiments, the dosage over the 24 hour period may be in the range of about 2.0 mg to about 4.0 mg. In various embodiments, the terlipressin is administered as a continuous intravenous (IV) drip lasting from about 4 hours to about 6 hours, and comprising a dosage of about 0.5 mg to about 2.0 mg. In various embodiments, the terlipressin dosage is not given as a bolus.

In various embodiments, the terlipressin dosage is given as a bolus injection. In one or more embodiments, a patient having HRS-1 and low MAP may be administered an effective dose of terlipressin by IV bolus injection, such that the dose is sufficient to yield an increase in MAP and decrease in heart rate in the patient. The administration of the effective dose of terlipressin may result in increased survival of the patient.

In one or more embodiments, a patient having HRS-1 and low MAP may be administered an effective dose of terlipressin to a patient by IV bolus injection, where the dose is sufficient to yield an increase in the patient's diastolic, systolic and MAP, and decrease in heart rate in the patient. The administration of the effective dose of terlipressin may result in increased survival of the patient.

In some embodiments, the increase in MAP may be about 1 mmHg to about 20 mmHg. In at least one example, the estimated maximum effect for MAP for the dose may be an increase of about 16.2 mmHg. In some embodiments, the decrease in heart rate may be a decrease of about 1 beat/minute to about 15 beats/minute. In at least one example, the estimated maximum effect for heart rate may be a decrease of 10.6 beats/minute. An increase in the diastolic, systolic and MAP, and decrease in heart rate may be evident within 5 minutes after dosing and may be maintained for at least 6 hours after dosing. The maximum change in blood pressure and heart rate may occur 1.2 to 2 hours after dosing, which may be the same time of maximum lysine-vasopressin plasma concentrations.

In one or more embodiments, the terlipressin dosage may be a dosage of about 0.5 mg to about 2.0 mg administered intravenously about every 4 to 12 hours as a slow bolus injection over about 1 to 5 minutes. In some embodiments, the dose may be administered about every 6 hours by IV bolus injection over about 2 minutes. In one or more embodiments, the terlipressin dosage is about 1 mg terlipressin acetate administered intravenously about every 6 hours as a slow bolus injection over about 2 minutes.

In an embodiment, the terlipressin administered may be terlipressin acetate. The terlipressin acetate dosage may be administered to the patient in the range of about 0.5 mg to about 4.0 mg. In various examples, the terlipressin acetate dosage may be about 0.5 mg, about 1 mg, about 1.5 mg, about 2 mg, about 2.5 mg, about 3 mg, about 3.5 mg, or about 4 mg. In some examples, the terlipressin dosage may be about 0.85 mg or about 1 mg terlipressin acetate. In other examples, the terlipressin acetate may be administered at a dosage of about 1 mg to about 2 mg. In at least one example, the initial dosage may be about 1.0 mg terlipressin acetate (i.e. 0.85 mg terlipressin) and may be increased to a dosage of about 2 mg terlipressin acetate.

The terlipressin may be prepared for injection as a white to off-white lyophilized powder in a single-dose vial for reconstitution at a dosage of 0.85 mg terlipressin (equivalent to 1 mg terlipressin acetate). In some embodiments, the terlipressin acetate dosage may be given at an initial dose of about 0.5 mg or about 1 mg. In at least one example, dosing may be initiated with 1 mg terlipressin acetate. In other embodiments, the terlipressin dosage may be modified after a period of time administering the initial dose. In at least one example, the modified dosage may be about 2 mg terlipressin acetate.

The dosage may be administered about every 4 hours, about every 5 hours, about every 6 hours, about every 7 hours, about every 8 hours, about every 9 hours, about every 10 hours, about every 11 hours, or about every 12 hours by slow IV bolus injection. In at least one example, the dosage may be administered about every 6 hours by slow IV bolus injection. The bolus injection may be given over about 1 minute, about 2 minutes, about 3 minutes, about 4 minutes, or about 5 minutes. In at least one example, the bolus injection may be given over about 2 minutes.

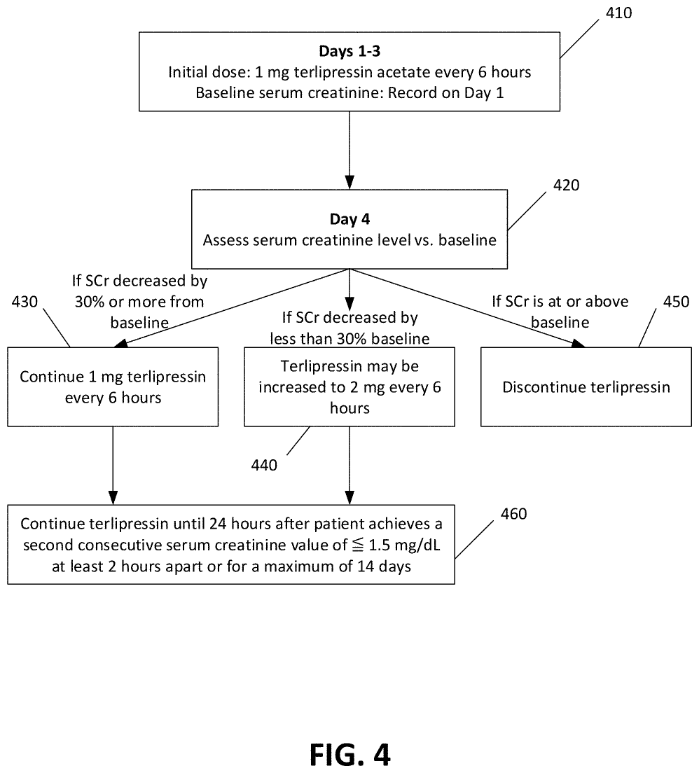

An aspect of the present disclosure relates to methods of treating and/or reversing HRS-1. As shown in FIG. 4, an exemplary embodiment of a method of treating an adult patient with HRS-1 via an embodiment of a terlipressin treatment protocol.

At step 410, in some embodiments, a baseline serum creatinine level may be measured before administration of terlipressin on day 1. Then, an initial dose of terlipressin may be administered to the patient with HRS-1. In an example, the initial dose of terlipressin may about 0.5 mg to about 1.0 mg terlipressin acetate, and it may be administered every 6 hours for about 1-3 days. In at least one example, the initial dosage may be about 1.0 mg terlipressin acetate (i.e. 0.85 mg terlipressin).

At step 420, on day 4±1 day of administration (e.g. after a minimum of 10 doses), the serum creatinine level may be assessed and compared to the baseline level. In various embodiments, the patient that is being administered the terlipressin is assessed at least once during days 1 to 4±1 day of administration to determine if the patient is responding to the terlipressin. In various embodiments, the patient may be tested once at the end of about 3 or 4 days of administration of the terlipressin. In some examples, the serum creatinine level may be continually assessed (e.g. daily) until administration is discontinued. In various embodiments, the dosage administered to the patient may be adjusted based upon the measured serum creatinine level(s). In various embodiments, a patient being administered terlipressin may have their serum creatinine levels monitored for the entire time period that the patient is receiving terlipressin. In one or more embodiments, the patient's serum creatinine level may be tested every day, or every other day, or every third day, or every fourth day, to confirm that the patient is still responding positively to the terlipressin treatment.

In some embodiments, the terlipressin may be administered to the patient for up to 4 days, wherein the patient may be tested each day of the four days to determine whether the patient is responding to the terlipressin treatment. In various embodiments, a response to the terlipressin treatment may be indicated by a change in the patient's serum creatinine levels, where indication may be a reduction in SCr of at least 25% from baseline. In various embodiments, the terlipressin may be administered for at least 4 days.

The serum creatinine levels may be measured by any of the methods known in the art, for example, the Jaffe reaction using alkaline picrate. The GFR may be measured directly by clearance studies of exogenous markers, such as inulin, iohexol, iothalamate, and Cr51-EDTA, by non-invasive detection of the change in patient levels of a fluorescent GFR tracer agent, or by estimated glomerular filtration rate (eGFR) using creatinine testing methods that are traceable to a reference method based on isotope dilution-mass spectrometry (IDMS).

In various embodiments, the patient's creatinine levels are assessed to determine if there has been a reduction in the patient's serum creatinine, where a reduction in serum creatinine levels of about 1.0 mg/dL or greater, or in the range of about 1.0 mg/dL to about 2.0 mg/dL, or a reduction of about 1.7 mg/dL from the patient's initial baseline value indicates an improvement in renal function and that the patient is responding to the terlipressin. In some examples, the assessed serum creatinine level may be 30% or more less than the baseline serum creatinine level, may be between 1% and 29% less than the baseline serum creatinine level, or may be 0% or greater than the baseline serum creatinine level. At steps 430, 440, and 450, a modified dosage of terlipressin may then be administered based on the comparison of the assessed serum creatinine level at day 4±1 day and the baseline serum creatinine level.

At step 430, if the assessed SCr level decreased by 30% or more from the baseline SCr level at day 4±1 day, the about 0.5 mg to about 1.0 mg dosage of terlipressin may be continued to be administered to the patient every 6 hours. For example, the modified dosage may be the same as the initial dosage (e.g. 0.5 mg to 1.0 mg) if the assessed SCr level decreased by 30% or more from the baseline SCr level.

In some embodiments, the amount of serum creatinine change is determined after 4 days of treatment with terlipressin, and the treatment with terlipressin continued if the serum creatinine level has improved. In various embodiments, a sufficient improvement in serum creatinine levels after 4 days of treatment is indicated by a decrease of at least 1.0 mg/dL in serum creatinine level, or a decrease of about 1.7 mg/dL in serum creatinine level.

In some embodiments, if the patient exhibits improvement over the previous 1 to 4 days, they may receive terlipressin for additional days until they reach the baseline SCr value<1.5 mg/dL. In various embodiments, the patient receives terlipressin for an additional about 1 day to about 10 days, about 3 days to about 4 days, about 3 days to about 6 days, about 3 days to about 8 days, about 3 days to about 10 days, or about 3 days to about 12 days if improvement was exhibited over the previous 1 to 4 days. In various embodiments, the patient receives terlipressin for an additional 3 days to 4 days if improvement was exhibited over the previous 1 to 4 days.

At step 440, if the assessed SCr level has decreased, but by less than 30% from the baseline level at day 4±1 day, the dosage of terlipressin may be increased to about 1.0 mg to about 2.0 mg about every 6 hours. For example, the modified dosage may be about 0.1 mg to about 2.0 mg of terlipressin acetate about every 6 hours (±30 min) (8 mg/day) if the assessed SCr level has decreased, but by less than 30% from the baseline level. In at least one example, the modified dosage may be 2 mg terlipressin acetate. The assessed dose may not be increased from the initial dose if the subject had coronary artery disease; or in the clinical setting of circulatory overload, pulmonary edema, or treatment-refractory bronchospasm. In various embodiments, if dosing was interrupted due to a non-ischemic adverse event, terlipressin may be restarted at the same or lower dose (i.e., 0.5 to 1 mg q6h).

At step 450, if the assessed SCr level is at or above the baseline SCr level at day 4±1 day, the administration of terlipressin may be discontinued. For example, the modified dosage may be a discontinuation of administering terlipressin if the assessed SCr level is at or above the baseline SCr level. Management of adverse reactions may include temporary dose reduction or interruption. Terlipressin may be given at a lower dose (e.g. 0.5 mg or 1 mg) or at a less frequent dosing interval (e.g. 8 to 12 hours). Upon resolution/improvement of adverse reactions, terlipressin may be resumed at the same or lower dose. If severe adverse reactions persist or recur following dose adjustment, the administration of terlipressin may be permanently discontinued.

At step 460, administration of terlipressin may be continued until 24 hours after the patient achieves a second consecutive serum creatinine value of <1.5 mg/dL at least 2 hours apart or for a maximum of 14 days. In various embodiments, the dosage may be repeated about every four to six hours for a time period of one or more days until the patient shows recovery, or until the patient no longer shows improvement. In various embodiments, the duration of treatment of a patient with terlipressin may be 1 to 14 days. In various embodiments, the terlipressin may be administered for at least 4 days. In various embodiments, the patient is administered terlipressin for up to about 14 days unless the patient experiences an adverse event. In various embodiments, the terlipressin may be administered for at least 3 days, at least 4 days, at least 5 days, at least 6 days, at least 7 days, at least 8 days, at least 9 days, at least 10 days, at least 11 days, at least 12 days, at least 13 days, or at least 14 days. In some examples, the terlipressin may be administered to the patient for a time period in the range of about 2 days to about 14 days, or for a time period in the range of about 4 days to about 8 days. In various embodiments, the time period is in the range of about 7 days. In various embodiments, the terlipressin treatment may be continued until there is a complete response.

In various embodiments, the administration of terlipressin to the patient is continued for an additional 3 days to 12 days beyond the initial 4 days if the patient exhibits a decrease in the serum creatinine level. In various embodiments, administration of terlipressin to the patient may be continued until at least one SCr value<1.5 mg/dL is obtained (i.e. HRS reversal). In some embodiments, administration of terlipressin to the patient may be continued until at least two SCr values of mg/dL are obtained at least 48 hours apart (i.e. verified HRS reversal). In various embodiments, the duration of treatment may be extended to a maximum of 15 days or 16 days if HRS reversal was first achieved on days 13 or 14, respectively. In various embodiments, the duration of treatment of a patient with terlipressin may be 1 to 28 days. In various embodiments, a decrease in the serum creatinine level may be indicated by a reduction in SCr of at least 1% or of at least 5% or at least 10% or at least 15% or at least 20% or at least 25% from baseline.

In various embodiments, the patient may experience HRS reversal about 4 to about 90 days after initiating administration of terlipressin. In additional embodiments, the patient may experience verified HRS reversal about 4 to about 90 days after initiating administration of terlipressin. In one or more embodiments, reversal of HRS is indicated by a decrease in SCr level to ≤1.5 mg/dl, and verified reversal of HRS is defined as two SCr values of ≤1.5 mg/dL at least 48 hours apart.

In an embodiment, the patient may have increased overall survival as compared to placebo. For example, a patient with a low baseline MAP treated with terlipressin may have a greater likelihood of surviving as compared to placebo. In some embodiments, the patient may have an increased median number of days of overall survival after initiating administration of terlipressin as compared to placebo. In some embodiments, the patient may have an increased overall survival at about 90 days after initiating administration of terlipressin as compared to placebo. For example, the patient may have a 50% to 185% increase in overall survival after initiating administration of terlipressin as compared to placebo. In some embodiments, the patient may have similar overall survival at about 90 days after initiating administration of terlipressin as compared to patients with HRS-1 but a baseline MAP 65 mmHg.

In an embodiment, the patient may have increased transplant-free survival as compared to placebo. For example, a patient with a low baseline MAP treated with terlipressin may have a greater likelihood of surviving and being transplant-free as compared to placebo. In some embodiments, the patient may have an increased median number of days transplant-free survival after initiating administration of terlipressin as compared to placebo. In other embodiments, the patient may have an increased transplant-free survival at about 90 days after initiating administration of terlipressin as compared to placebo. For example, the patient may a 30% to 145% increase in transplant-free survival after initiating administration of terlipressin as compared to placebo. In other embodiments, the patient may have similar transplant-free survival at about 90 days after initiating administration of terlipressin as compared to patients with HRS-1 but a baseline MAP 65 mmHg.

In one or more embodiments, the patient may have been administered albumin prior to beginning the terlipressin treatment protocol, and/or prior to the determination that the patient has HRS-1 or low baseline MAP. In various embodiments, albumin may be administered to a patient 7 days to 2 days before starting administration of terlipressin to the patient. In various embodiments, the albumin treatment comprises administering 1 gram albumin per 1 kg of patient weight up to a maximum of 100 g per day of albumin to a patient. In various embodiments, albumin may be administered in the range of about 20 g/day to about 50 g/day, where the albumin may be administered for the time period that the patient is administered terlipressin.

A non-limiting embodiment of a method of treating HRS-1 patients exhibiting low baseline MAP with terlipressin comprises administering to a patient in need of such treatment a dosage of terlipressin in the range of 2.0 mg to 12.0 mg per day for 1 to 28 days, or in the range of 2.0 mg to 4.0 mg per day for 1 to 7 days, wherein the dosage may be administered as a continuous IV feed or as a slow bolus injection.

Embodiments of the present disclosure also relate to treating patients with HRS-1 and low baseline MAP with one dose of terlipressin every six hours, where the dose is in the range of about 0.5 mg to 2.0 mg, for 3 to 8 days to achieve reversal of the HRS-1.

Embodiments of the present disclosure also relate to initiating terlipressin treatment within 48 hours of determining that a patient is presenting with HRS-1 and MAP <65 mmHg, but without sepsis, septic shock, or uncontrolled infection.

Another aspect of the present disclosure relates to a method of distributing a pharmaceutical product.

In one or more embodiments, the method of distributing comprises supplying terlipressin to a medical provider, where the medical provider may be responsible for treating a patient suffering from type 1 hepatorenal syndrome. In various embodiments, the patient does not have overt sepsis, septic shock, or uncontrolled infection. In various embodiments, the method includes providing a recommendation to the medical provider to treat the patient suffering from type 1 hepatorenal syndrome that does not have overt sepsis, septic shock, or uncontrolled infection and having a baseline MAP of <65 mmHg, with terlipressin in an amount effective to improve overall survival, transplant free-survival, and/or reduce SCr. In one or more embodiments, the medical provider follows the recommendation and administers a treatment to the patient suffering from HRS-1, but not overt sepsis, septic shock, or uncontrolled infection and having a baseline MAP of <65 mmHg, with terlipressin in an amount effective to improve overall survival, transplant free-survival, and/or reduce SCr.

### EXAMPLES

### Example 1

A randomized, placebo-controlled, double-blind study was conducted to evaluate the efficacy of terlipressin in HRS type 1. The objective of the study was to determine the efficacy and safety of intravenous terlipressin compared with placebo in the treatment of adult patients with HRS type1 receiving intravenous albumin. Men and women aged 18 years or older having cirrhosis, ascites, and a diagnosis of HRS type 1 based on the 2007 International Ascites Club (IAC) diagnostic criteria (Salerno F, Gerbes A, Gines P, Wong F, Arroyo V., Diagnosis, prevention and treatment of hepatorenal syndrome in cirrhosis, Gut. 2007; 56:1310-1318) were eligible for participation. Patients with an SCr level>2.5 mg/dL and either a doubling of SCr within 2 weeks or a change in SCr levels over time indicating a trajectory with a slope equal to or greater than that of a doubling within 2 weeks were enrolled. Patients with uncontrolled infection, sepsis, or septic shock were excluded.

Baseline MAP was used to dichotomize 307 patients with HRS-1 into 2 groups stratified according to baseline MAP: <65 mmHg and 65 mmHg, with 50 patients having a baseline MAP <65 mmHg and 257 patients having a baseline MAP 65 mmHg.

Between-group comparisons for HRS reversal were conducted within each MAP group using the Fisher exact test; P values comparing survival estimates were calculated using a two sample log-rank test (randomization was stratified by SCr, <3.6 mg/dL or mg/dL) and alcoholic hepatitis (present or not).

Exclusion criteria were intended to produce a patient sample limited to individuals with functional renal impairment secondary to cirrhosis and ascites, who could safely be administered terlipressin and who could be expected to survive through the active study period.

Evaluation of patients with asymptomatic hypotension demonstrated that asymptomatic hypotension is not related to the presence of systemic inflammatory response syndrome (SIRS) or alcoholic hepatitis, as seen in Table 1 below.

TABLE 1

| | | |
|---|---|---|
| SIRS | EtOH hepatitis | Female |

| | | | | | |
|---|---|---|---|---|---|
| <65 mmHg | 7 | (22%) | 7 | (22%) | 52% |
| N = 32 | | | | | |
| ≥65 mmHg | 51 | (31%) | 38 | (23%) | 32% |
| N = 164 | | | | | |

In the MAP <65 mmHg group, mean (SD) duration of exposure to study treatment was 4.7 (3.54) days with terlipressin versus 4.5 (2.77) days with placebo. In the MAP 65 mm Hg group, the respective mean (SD) duration of exposure was 6.0 (4.27) versus 6.3 (3.99).

### Example 2

The two patient groups were compared to placebo for HRS reversal, confirmed or verified HRS reversal, change in SCr, and change in MAP.

Patients identified as having HRS-1 and a baseline MAP of <65 mmHg on a terlipressin treatment protocol exhibited an increase in confirmed reversal of HRS (25%% vs. 8%, p<0.247), HRS reversal (24% vs. 8%, p<0.247), renal function (change from baseline in SCr, mg/dL, −0.8 vs. 0.2, p<0.0001), and MAP (change from baseline MAP, mmHg, 14.4 vs. 3.4, p<0.001) compared to placebo. In contrast, in the group of patients having HRS-1 and baseline MAP 65 mmHg, confirmed reversal of HRS vs. placebo was 24.2% vs. 14%, HRS reversal vs. placebo was 28.1% vs. 15.5%, renal function change vs. placebo was −0.9 vs. −0.6 mg/dL, and MAP change from baseline vs. placebo was 2.0 vs. −2.4 mmHg.

Rates of HRS reversal among patients receiving terlipressin were similar between the MAP <65 mmHg and 65 mmHg groups. The proportion of patients with HRS reversal in the MAP 65 mmHg group was significantly higher among patients receiving terlipressin than among those receiving placebo. Improvement in SCr from baseline to end of treatment was significantly greater with terlipressin than with placebo in both MAP groups. However, the degree of improvement was lower in the MAP 65 mmHg group.

TABLE 2
HRS Reversal, Change in SCr, and Change in MAP

| | Baseline MAP < 65 mmHg | | | | | Baseline MAP ≥ 65 mmHg | | | | |
|---|---|---|---|---|---|---|---|---|---|---|
| | Terlipressin | | Placebo | | | Terlipressin | | Placebo | | |
| | N = 25 | | N = 25 | | P-Value | N = 128 | | N = 129 | | P-Value |
| HRS Reversal | 6 | (24%) | 2 | (8%) | 0.2467 | 36 | (28.1%) | 20 | (15.5%) | 0.0159 |
| Confirmed | 6 | (24%) | 2 | (8%) | 0.2467 | 31 | (24.2%) | 18 | (14%) | 0.0399 |
| HRS Reversal | | | | | | | | | | |
| LS mean (SE) | −0.8 | (0.20) | 0.2 | (0.19) | 0.0001 | −0.9 | (0.11) | −0.6 | (0.10) | 0.042 |
| change in SCr, | | | | | | | | | | |
| baseline to | | | | | | | | | | |
| EOT, mg/dL | | | | | | | | | | |
| LS mean (SE) | 14.4 | (1.54) | 3.4 | (1.44) | <0.001 | 2.0 | (0.81) | −2.4 | (0.81) | <0.001 |
| change in | | | | | | | | | | |
| MAP, baseline | | | | | | | | | | |
| to EOT, | | | | | | | | | | |
| mmHg | | | | | | | | | | |

### Example 3

The two patient groups were compared to placebo for overall survival and transplant free survival.

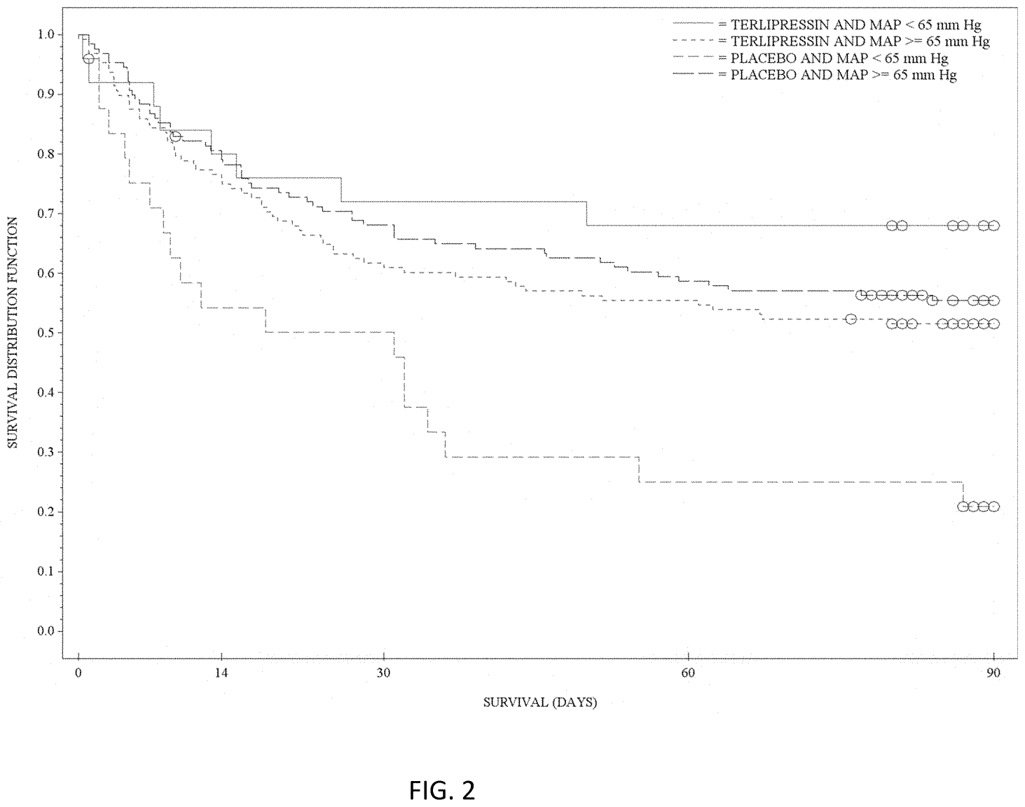

Patients with HRS-1 and low MAP treated with terlipressin showed an improved overall survival (OS) rate at 90 days compared to placebo (68% vs. 24%; P=0.005). Moreover, patients with HRS-1 and low MAP showed an improved overall survival rate at 90 days compared to patients that were suffering from HRS-1, but did not have low MAP when both groups were treated with terlipressin (68% vs. 51.6%). Whereas no difference in OS at 90 days was observed between terlipressin and placebo in the MAP 65 mm Hg group (51.6% vs. 55.8%; P=0.429). FIG. 2 shows the overall survival of the two patient groups, comparing the treatment groups and placebo.

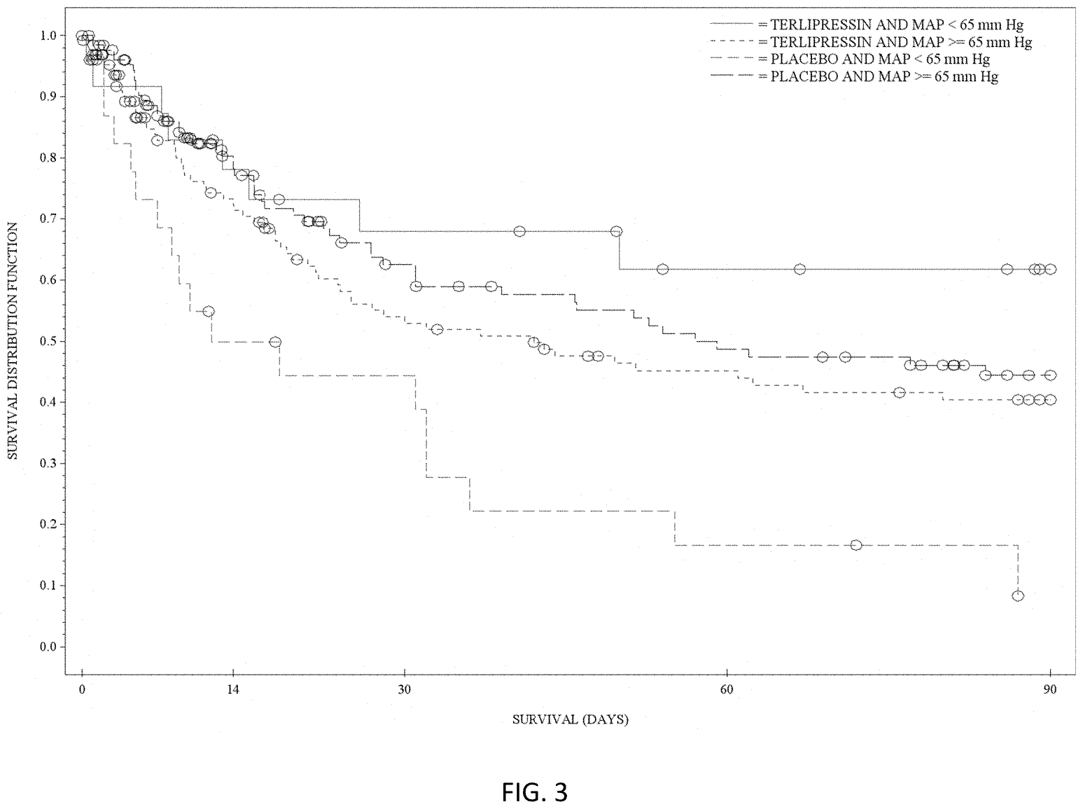

Further, patients with HRS-1 and low MAP treated with terlipressin showed an improved transplant-free survival (TFS) rate compared to placebo (68% vs. 28%; P=0.015). In the treatment groups, patients with HRS-1 and low MAP showed an improved transplant-free survival rate compared to patients that were suffering from HRS-1, but did not have low MAP (68% vs. 52%). Whereas in the MAP 65 mmHg group, no difference in TFS at 90 days was observed between terlipressin and placebo (52.3% vs. 58.9%; P=0.291). FIG. 3 shows the transplant-free survival of the two patient groups, comparing the treatment groups and placebo.

These results are further detailed in Table 3 below.

TABLE 3
Overall Survival and Transplant-Free Survival by Baseline MAP

| | Baseline MAP < 65 mmHg | | Baseline MAP ≥ 65 mmHg | |
|---|---|---|---|---|
| | Terlipressin | Placebo | Terlipressin | Placebo |
| | N = 25 | N = 25 | N = 128 | N = 129 |
| Overall | 17 (68%) | 6 (24%) | 66 (51.6%) | 72 (55.8%) |
| survival, | | | | |
| alive at | | | | |
| day 90 | | | | |
| Transplant- | 17 (68%) | 7 (28%) | 67 (52.3%) | 76 (58.9%) |
| free survival, | | | | |
| alive at day 90 | | | | |

Treatment with terlipressin was associated with a considerable improvement in OS and TFS in patients with HRS-1 and a baseline MAP <65 mm Hg. This effect appears to be independent of HRS-1 reversal and may be related to a marked improvement in SCr from baseline to EOT in this group. Patients in this group who received terlipressin also experienced a significant improvement in MAP from baseline to EOT compared with patients who received placebo.

These results indicate that the presence of a baseline MAP of <65 mmHg indicates that the patient is more likely to have a positive response to treatment with terlipressin as compared to placebo.

It was also recognized that patients with decompensated liver disease frequently have low MAP in the absence of uncontrolled infection or sepsis, and that the presence of low MAP is associated with a poor prognosis. Therefore, it was surprising that patients with low MAP significantly improved overall survival and transplant-free survival.

### Example 4

A randomized, placebo-controlled, double-blind study was conducted to evaluate the efficacy of terlipressin in HRS type 1. The objective of the study was to characterize the efficacy and safety of terlipressin plus albumin versus albumin alone for the treatment of HRS-1 in patients with well-defined HRS-1. The study used the similar inclusion and exclusion criteria as described in Example 1.

300 subjects were enrolled in the study. Of the 300 subjects, 199 were randomized to terlipressin and 101 to placebo (albumin alone). Demographic and BL clinical characteristics were similar between treatment groups. For example, the two treatment groups had similar average age, weight, height, sex distribution, ethnicity distribution, race distribution, presence of alcoholic hepatitis, baseline serum creatinine, large volume paracentesis (LVP) randomization strata, baseline model end stage liver disease (MELD) score, baseline Child-Pugh score, baseline white blood cell count, baseline bilirubin, baseline mean arterial pressure (MAP), baseline heart rate, baseline blood urea nitrogen (BUN), baseline bicarbonate (HCO3) or carbon Dioxide (CO2), baseline temperature, baseline respiratory rate, baseline acute on chronic liver failure (ACLF) grade, baseline chronic liver failure-sepsis organ failure assessment (CLIF-SOFA) score and presence of prior conditions/treatments such as esophageal variceal hemorrhage (EVH) banding, pneumonia, urinary tract infection (UTI), spontaneous bacterial peritonitis (SBP), and receipt of albumin. The proportion of patients in each group who underwent LT was 23.1% for terlipressin and 28.7% for placebo.

Thirty three patients had a baseline MAP of <65 mmHg, 24 of which received terlipressin treatment and 9 received placebo. The results of the overall survival of the patients with a baseline MAP of <65 mmHg are shown in Table 4 and the results of the transplant-free survival of the patients with a baseline MAP of <65 mmHg are shown in Table 5 below.

TABLE 4
Overall Survival

| Baseline MAP < 65 mmHg | |
|---|---|
| Terlipressin | Placebo |

| | N | Parameter | N | Parameter |
|---|---|---|---|---|
| Overall Survival up to | | | | |
| 90 Days | | | | |
| Survival Estimate | 24 | 0.497 | 9 | 0.250 |
| Median Days of Survival | 24 | 72.0 | 9 | 27.0 |
| Alive at Day 90 (n, %) | 24 | 12 (50.0) | 9 | 3 (33.3) |

TABLE 5
Transplant-free Survival

| | Baseline MAP < 65 mmHg | | | |
|---|---|---|---|---|
| | Terlipressin | | Placebo | |
| | N | Parameter | N | Parameter |
| Transplant-Free Survival up to | | | | |
| 90 Days | | | | |
| Survival Estimate | 24 | 0.194 | 9 | 0.125 |
| Median Days of Survival | 24 | 20.0 | 9 | 15.5 |
| Alive and Transplant-Free at | 24 | 5 (20.8) | 9 | 2 (22.2) |
| Day 90 (n, %) | | | | |

Although the disclosure herein has been described with reference to particular embodiments, it is to be understood that these embodiments are merely illustrative of the principles and applications of the present disclosure. It will be apparent to those skilled in the art that various modifications and variations can be made to the devices, systems, and methods of the present disclosure without departing from the spirit and scope of the disclosure. Thus, it is intended that the present disclosure include modifications and variations that are within the scope of the appended claims and their equivalents.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

What is claimed is:

1.-35. (canceled)

36. A method of increasing survival of a patient having type 1 hepatorenal syndrome (HRS-1) and low mean arterial pressure (MAP), the method comprising:
administering an effective dose of terlipressin to a patient in need thereof every 6 hours by intravenous (IV) bolus injection over 2 minutes,
wherein the dose is sufficient to yield an increase in MAP and decrease in heart rate in the patient.

37. The method of claim 36, wherein the estimated maximum effect for MAP for the dose is an increase of 16.2 mmHg.

38. The method of claim 36, wherein the estimated maximum effect for heart rate is a decrease of 10.6 beats/minute.

39. The method of claim 36, wherein the terlipressin administered is terlipressin acetate.

40. The method of claim 39, wherein the effective dose is about 0.5 mg to about 2 mg terlipressin acetate.

41. The method of claim 36, further comprising determining if the patient has a reduction in serum creatinine level during an initial 1 to 4 days of terlipressin administration.

42. The method of claim 41, further comprising discontinuing administration of terlipressin to the patient if the patient does not show a reduction in serum creatinine level during the initial 1 to 4 days of terlipressin administration.

43. The method of claim 41, further comprising continuing administration of terlipressin to the patient for an additional 3 to 12 days if the patient shows a reduction in serum creatinine level during the initial 1 to 4 days of terlipressin administration.

44. The method of claim 43, further comprising increasing the dose of terlipressin from an initial dose to a modified dose after the initial 1 to 4 days of terlipressin administration.

45. The method of claim 44, wherein the initial dose is about 0.5 mg to about 1 mg terlipressin acetate and the modified dose is about 1 mg to about 2 mg terlipressin acetate.

46. A method of increasing survival of a patient having type 1 hepatorenal syndrome (HRS-1) and low mean arterial pressure (MAP), the method comprising:
administering an effective dose of terlipressin to a patient in need thereof every 6 hours by intravenous (IV) bolus injection over 2 minutes,
wherein the dose is sufficient to yield an increase in the diastolic, systolic and MAP, and decrease in heart rate in the patient.

47. The method of claim 46, wherein the estimated maximum effect for MAP for the dose is an increase of 16.2 mmHg.

48. The method of claim 46, wherein the estimated maximum effect for heart rate is a decrease of 10.6 beats/minute.

49. The method of claim 46, wherein the terlipressin administered is terlipressin acetate.

50. The method of claim 49, wherein the effective dose is about 1 mg to about 2 mg terlipressin acetate.

51. The method of claim 46, further comprising determining if the patient has a reduction in serum creatinine level during an initial 1 to 4 days of terlipressin administration.

52. The method of claim 51, further comprising discontinuing administration of terlipressin to the patient if the patient does not show a reduction in serum creatinine level during the initial 1 to 4 days of terlipressin administration.

53. The method of claim 51, further comprising continuing administration of terlipressin to the patient for an additional 3 to 12 days if the patient shows a reduction in serum creatinine level during the initial 1 to 4 days of terlipressin administration.

54. The method of claim 53, further comprising increasing the dose of terlipressin from an initial dose to a modified dose after the initial 1 to 4 days of terlipressin administration.

55. The method of claim 54, wherein the initial dose is about 0.5 mg to about 1 mg terlipressin acetate and the modified dose is about 1 mg to about 2 mg terlipressin acetate.

56. A method of increasing survival of a patient having type 1 hepatorenal syndrome (HRS-1) and low mean arterial pressure (MAP), the method comprising:
administering an effective dose of about 0.5 mg to about 2 mg terlipressin acetate to a patient in need thereof about every 4 to 10 hours by intravenous (IV) bolus injection over about 1 to 5 minutes,
wherein the dose is sufficient to yield an increase in MAP and decrease in heart rate in the patient.

57. The method of claim 56, wherein the dose is further sufficient to yield an increase in the diastolic and systolic pressure.

58. The method of claim 56, wherein the estimated maximum effect for MAP for the dose is an increase of 16.2 mmHg.

59. The method of claim 56, wherein the estimated maximum effect for heart rate is a decrease of 10.6 beats/minute.

60. The method of claim 56, further comprising determining if the patient has a reduction in serum creatinine level during an initial 1 to 4 days of terlipressin acetate administration.

61. The method of claim 60, further comprising discontinuing administration of terlipressin acetate to the patient if the patient does not show a reduction in serum creatinine level during the initial 1 to 4 days of terlipressin acetate administration.

62. The method of claim 60, further comprising continuing administration of terlipressin acetate to the patient for an additional 3 to 12 days if the patient shows a reduction in serum creatinine level during the initial 1 to 4 days of terlipressin acetate administration.

63. The method of claim 62, further comprising increasing the dose of terlipressin acetate from an initial dose to a modified dose after the initial 1 to 4 days of terlipressin acetate administration.

64. The method of claim 63, wherein the initial dose is about 0.5 mg to about 1 mg terlipressin acetate and the modified dose is about 1 mg to about 2 mg terlipressin acetate.


SEQUENCE LISTING (1 sequence)

SEQ ID NO: 1 - protein, 12 aa, Artificial Sequence
  SYNTHESIZED
GKPCNQFYCGGG